(19) United States
(12) Patent Application Publication
(10) Pub. No.: US 2023/0279606 A1
(43) Pub. Date: Sep. 7, 2023

(54) LOW-FRICTION DRYING SYSTEM

(71) Applicants: Xuanjun LI, Wilmington, DE (US); Haier US Appliance Solutions, Inc., Wilmington, DE (US)

(72) Inventors: Dong Soo Shin, Seoul (KR); Taehoon Lim, Seoul (KR); Jaebong Lee, Seoul (KR); Xuanjun Li, Shanghai (CN); Kevin Farrelly Nolan, Louisville, KY (US)

(21) Appl. No.: 16/936,803

(22) PCT Filed: May 18, 2020
(86) PCT No.: PCT/CN2020/090806

§ 371 Date: Jul. 23, 2020

(51) Int. Cl. D06F 58/10; D06F 58/24; D06F 34/32; D06F 34/26; A61L 9/20

(52) U.S. Cl. CPC D06F 58/10; A61L 9/20; D06F 34/26; D06F 34/32; D06F 58/24; D06F 2103/32

(57) ABSTRACT

A drying system, as provided herein, may include a cabinet, an air outlet grate, an air inlet grate, a door, a vapor compression assembly, and an ultraviolet light source or tank capacitance circuit. The cabinet may define a static chamber along the airflow path for the receipt of articles therein. The air outlet grate may be upstream from the static chamber along the airflow path to direct air thereto. The air inlet grate may be downstream from the static chamber along the airflow path to direct air therefrom. The door may be attached to the cabinet to selectively restrict access to the static chamber. The vapor compression assembly may be attached to the cabinet and defining a refrigerant flow path spaced apart from the static chamber. The vapor compression assembly may include a compressor, an evaporator, and a condenser.

### CROSS REFERENCE TO RELATED APPLICATIONS

The present application is the National Stage Entry of and claims the benefit of priority under 35 U.S.C. § 371 to PCT Application Serial No. PCT/CN2020/09086 filed May 18, 2020 and entitled LOW-FRICTION DRYING SYSTEM, which is hereby incorporated by reference in its entirety.

### FIELD OF THE INVENTION

The present subject matter relates generally to dryer appliances, and more particularly to drying appliances for treating articles according to a low-friction cycle.

### BACKGROUND OF THE INVENTION

Dryer appliances typically include a cabinet with a drum mounted therein. In many dryer appliances, a motor rotates the drum during operation of the dryer appliance to tumble articles located within a chamber defined by the drum. Alternatively, dryer appliances with fixed drums have been used. Typical dryer appliances also generally include a heater assembly that passes heated air through the chamber of the drum in order to dry a load of moisture-laden articles disposed within the chamber. This internal air then passes from the chamber through a vent duct to an exhaust conduit, through which the air is exhausted from the dryer appliance. Typically, a blower (also known as an air handler) is used to flow the internal air from the vent duct to the exhaust duct. When operating, the blower may pull air through itself from the vent duct, and this air may then flow from the blower to the exhaust conduit.

Although typical dryer appliances may be suitable to treat many different types of articles, there are many types of delicate articles or materials (e.g., formed from or including cashmere, silk, elastane, etc.) that are not suitable for such dryer appliances. For instance, the high-friction environment of a rotating drum may distress or damage certain articles. Additionally or alternatively, the uneven heat supplied to a crumpled mass of articles within a typical dryer appliance may generate irregular creases, improperly-dried portions, or damage for some articles. Moreover, it may difficult to typical dryer appliances to determine how much moisture has been extracted from a given load.

Separately from or in addition to drying articles, some users may wish to use a dryer appliance to “freshen” certain articles. As an example, a user may wish to use the heat or airflow generated within a dryer appliance to kill various bacteria or viruses on an article. As another example, a user may wish to mitigate or remove odors from an article. Typical dryer appliances may, however, struggle to perform such duties, especially for relatively delicate articles.

As a result, dryer appliances or systems that can address one or more of the above issues would be useful. For instance, it may be advantageous to provide a dryer system having improved or detectable drying performance for (e.g., delicate) articles therein. Additional or alternatively, it may be advantageous to provide a dryer system having improved cleaning or odor-removal performance for (e.g., delicate) articles therein.

### BRIEF DESCRIPTION OF THE INVENTION

Aspects and advantages of the invention will be set forth in part in the following description, or may be obvious from the description, or may be learned through practice of the invention.

In one exemplary aspect of the present disclosure, a drying system is provided. The drying system may include a cabinet, an air outlet grate, an air inlet grate, a door, a vapor compression assembly, and an ultraviolet light source. The cabinet may define a static chamber along the airflow path for the receipt of articles therein. The air outlet grate may be upstream from the static chamber along the airflow path to direct air thereto. The air inlet grate may be downstream from the static chamber along the airflow path to direct air therefrom. The door may be attached to the cabinet to selectively restrict access to the static chamber. The vapor compression assembly may be attached to the cabinet and defining a refrigerant flow path spaced apart from the static chamber. The vapor compression assembly may include a compressor, an evaporator, and a condenser. The compressor may selectively compress refrigerant within the refrigerant flow path. The evaporator may be in fluid communication with the refrigerant flow path. The evaporator may be mounted along the airflow path between the air inlet grate and the air outlet grate. The condenser may be in fluid communication with the refrigerant flow path. The condenser may be mounted along the airflow path between the evaporator and the air outlet grate. The ultraviolet light source may be mounted along the airflow path between the air outlet grate and the evaporator.

In another exemplary aspect of the present disclosure, a drying system is provided. The drying system may include a cabinet, an air outlet grate, an air inlet grate, a door, a vapor compression assembly, a condensation path, a water tank, and a tank capacitance circuit. The cabinet may define a static chamber along the airflow path for the receipt of articles therein. The air outlet grate may be upstream from the static chamber along the airflow path to direct air thereto. The air inlet grate may be downstream from the static chamber along the airflow path to direct air therefrom. The door may be attached to the cabinet to selectively restrict access to the static chamber. The vapor compression assembly may be attached to the cabinet and defining a refrigerant flow path spaced apart from the static chamber. The vapor compression assembly may include a compressor, an evaporator, and a condenser. The compressor may selectively compress refrigerant within the refrigerant flow path. The evaporator may be in fluid communication with the refrigerant flow path. The evaporator may be mounted along the airflow path between the air inlet grate and the air outlet grate. The condenser may be in fluid communication with the refrigerant flow path. The condenser may be mounted along the airflow path between the evaporator and the air outlet grate. The condensation path may be disposed downstream from the evaporator outside of the refrigerant flow path to receive a liquid condensation therefrom. The condensation path may extend from a first end positioned below the evaporator to a second end positioned apart from the airflow path. The water tank may be positioned at the second end of the condensation path to receive the liquid condensation. The water tank may include a conductive sidewall plate extending from a bottom end to a top end. The tank capacitance circuit may be in electrical communication with the conductive sidewall plate.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides an elevation view of a dryer appliance according to exemplary embodiments of the present disclosure.

FIG. 2 provides a perspective view of the exemplary dryer appliance of FIG. 1.

FIG. 3 provides a perspective view of the exemplary dryer appliance of FIG. 1, wherein portions of the cabinet and casing have been removed for clarity.

FIG. 4 provides a schematic view of the exemplary dryer appliance of FIG. 1.

FIG. 5 provides a perspective view of a portion of the vapor compression assembly of the exemplary dryer appliance of FIG. 1.

FIG. 6 provides a perspective view of a dryer appliance according to exemplary embodiments of the present disclosure.

FIG. 7 provides a rear perspective view of a vapor compression assembly of a dryer appliance according to exemplary embodiments of the present disclosure.

FIG. 8 provides a side, schematic, plan view of a water tank of a dryer appliance according to exemplary embodiments of the present disclosure.

FIG. 9 provides a top, schematic, plan view of a door-detection assembly of a dryer appliance according to exemplary embodiments of the present disclosure.

FIG. 10 provides a top, schematic, plan view of a door-detection assembly of a dryer appliance according to other exemplary embodiments of the present disclosure.

FIG. 11 provides a top, schematic, plan view of a door-detection assembly of a dryer appliance according to yet other exemplary embodiments of the present disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the term “or” is generally intended to be inclusive (i.e., “A or B” is intended to mean “A or B or both”). The terms “first,” “second,” and “third” may be used interchangeably to distinguish one component from another and are not intended to signify location or importance of the individual components. The terms “upstream” and “downstream” refer to the relative flow direction with respect to fluid flow in a fluid pathway. For example, “upstream” refers to the flow direction from which the fluid flows, and “downstream” refers to the flow direction to which the fluid flows.

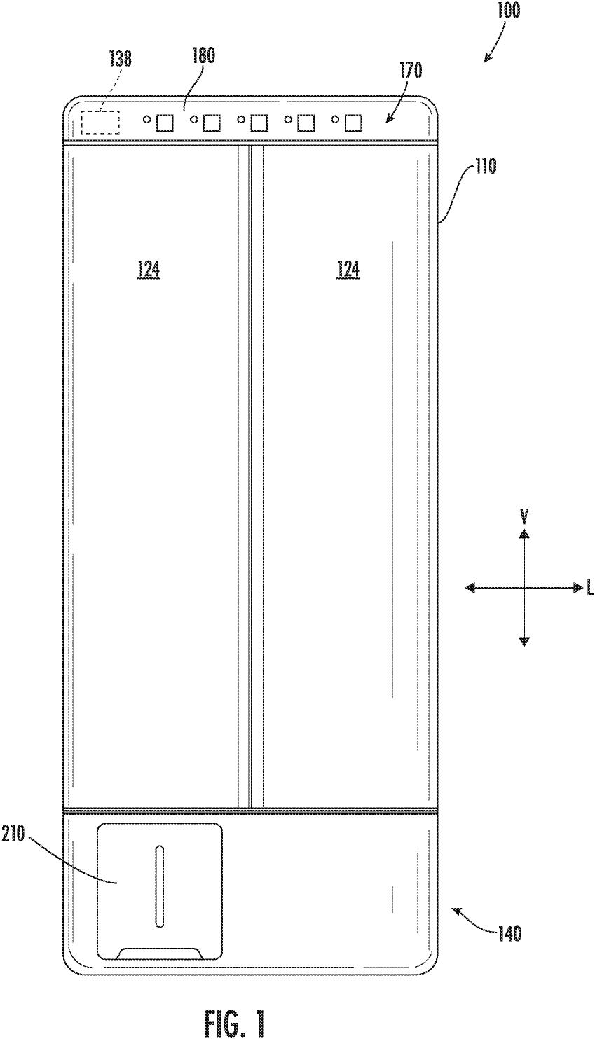

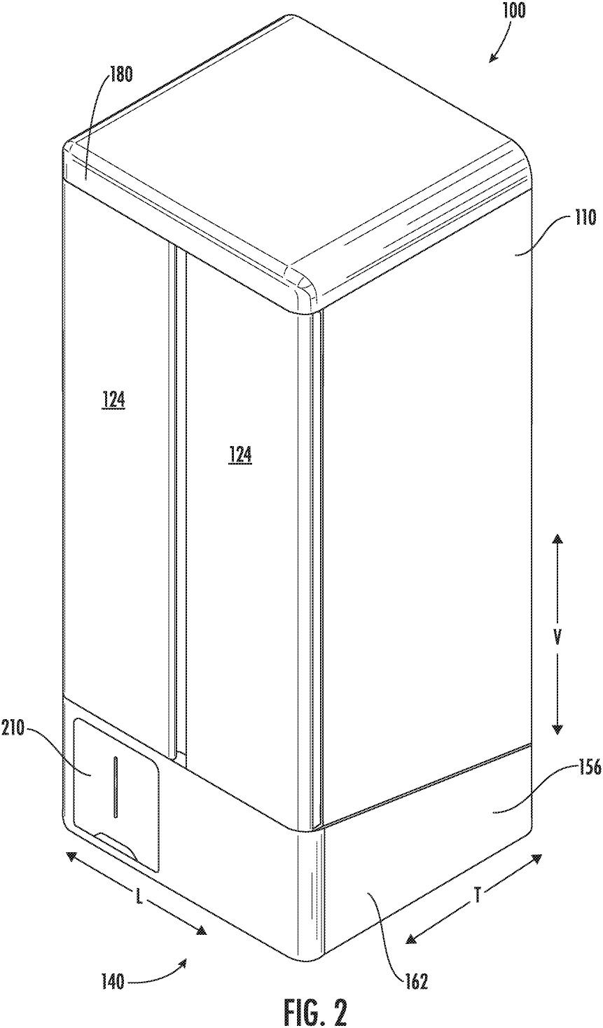

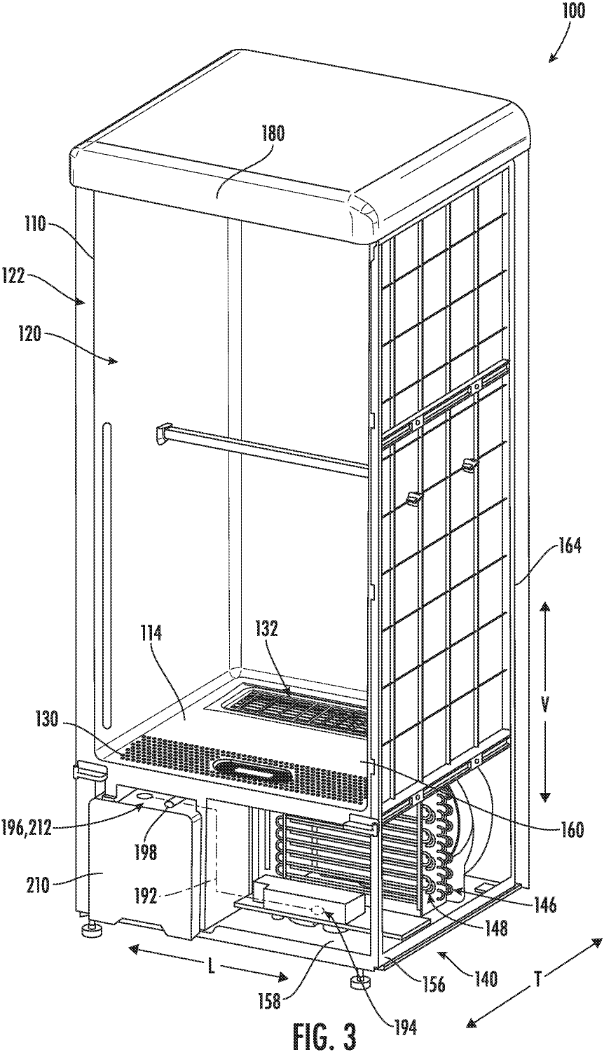

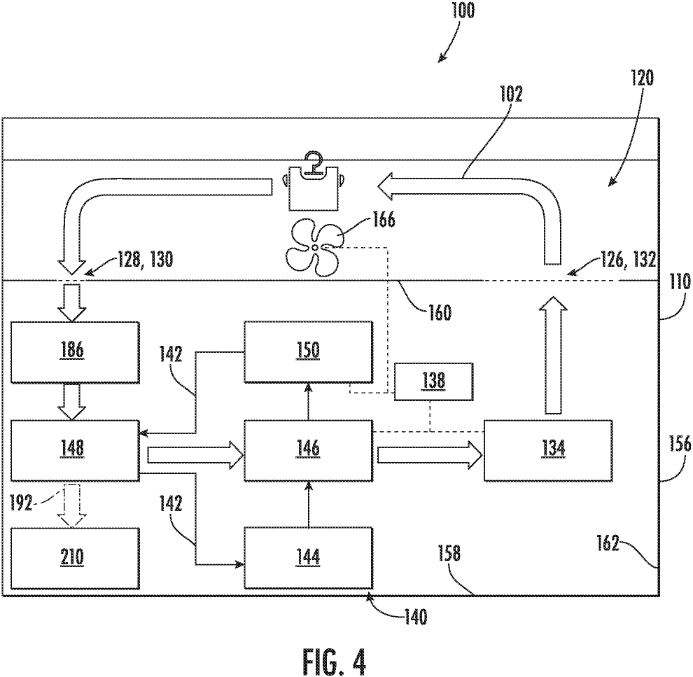

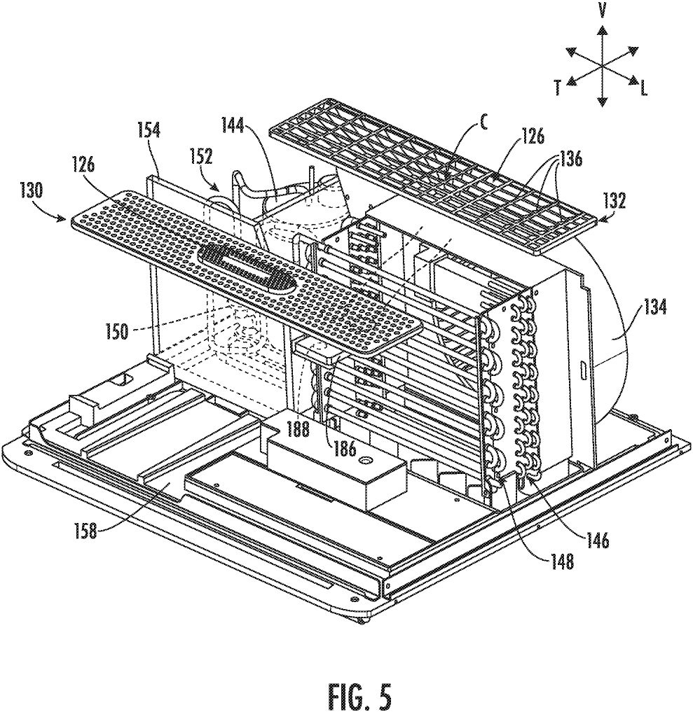

Turning now to the figures, FIGS. 1 through 4 provide various views of a dryer system or appliance 100 according to exemplary embodiments of the present disclosure. As shown, dryer appliance 100 generally defines a mutually-orthogonal vertical direction V, lateral direction L, and transverse direction T. Within dryer appliance 100, an airflow path 102 is defined (e.g., as a closed loop), as will be described in greater detail below.

In some embodiments, dryer appliance 100 includes a cabinet 110 having one or more internal walls, such as a lower internal wall (e.g., 114), upper internal wall, and side internal walls extending therebetween (e.g., along the vertical direction V). Together, the internal walls define a static chamber 120 for the receipt of clothing or articles therein. As shown, a chamber opening 122 is defined (e.g., at a front of cabinet 110) by the internal walls such that a user is able to insert or remove articles from static chamber 120 (e.g., along the transverse direction T). Optionally, one or more support members (e.g., bars, hooks, tabs, etc.) may be mounted within static chamber 120 to hold or support articles to be treated. Thus, articles within static chamber 120 may hang in place during operation of dryer appliance 100.

In some embodiments, one or more chamber doors 124 are attached to cabinet 110 (e.g., directly or indirectly) to selectively restrict access to static chamber 120. In particular, each chamber door 124 may move between a closed position (FIG. 1) and an open position (not pictured). In the closed position, chamber door 124 covers at least a portion of chamber opening 122 and thereby restricts access to static chamber 120. By contrast, in the open position, chamber door 124 is spaced apart from chamber opening 122 and thereby permits access to static chamber 120. In the illustrated embodiments, a pair of chamber doors 124 are pivotably attached to cabinet 110 at opposite lateral ends. Such a configuration may be referred to as a French door arrangement. Nonetheless, it is understood that, except as otherwise provided, the present disclosure is not limited to any particular configuration. Any suitable configuration of one or more doors is encompassed within the present disclosure.

Generally, static chamber 120 may be defined along an airflow path 102 defined by dryer appliance 100. As shown, one or more outlet apertures 126 are defined upstream from static chamber 120 while one or more inlet apertures 128 are defined downstream from static chamber 120. Thus, static chamber 120 is defined in fluid communication between at least one outlet aperture 126 and inlet at least one inlet aperture 128. In some embodiments, an air outlet grate 132 defines the one or more outlet apertures 126. Thus, relative to or along the airflow path 102, air outlet grate 132 is upstream from static chamber 120 to direct air to static chamber 120. In additional or alternative embodiments, an air inlet grate 130 defines the one or more inlet apertures 128. Thus, relative to or along the airflow path 102, an air inlet grate 130 is downstream from static chamber 120 to direct air from static chamber 120.

In certain embodiments, air inlet grate 130 is spaced apart from air outlet grate 132. For instance, air outlet grate 132 may positioned be rearward from air inlet grate 130 (e.g., along the transverse direction T). In particular, air outlet grate 132 may be distal to chamber opening 122 while air inlet grate 130 may be proximal to chamber opening 122. Optionally, air inlet grate 130 and air outlet grate 132 may be positioned in a parallel or common horizontal plane (i.e., a plane perpendicular to the vertical direction V). Additionally or alternatively, air inlet grate 130 and air outlet grate 132 may be positioned at a bottom end of static chamber 120. During operation, air may be directed generally upward through air outlet grate 132 into static chamber 120 (e.g., as motivated by a system fan 134) before at least of a portion of such air is redirected downward and out of static chamber 120 through air inlet grate 130.

In exemplary embodiments, air outlet grate 132 defines the outlet apertures 126 according to an expanding passage shape. Specifically, one or more internal guiding vanes 136 may be angled outward relative to a centerpoint C. In some such embodiments, air outlet grate 132 may define a laterally-expansive airflow passage along the vertical direction V. Thus, as illustrated, certain internal guiding vanes 136 may be positioned at a non-parallel angle relative to the vertical direction V. For instance, at least one vane 136 may define a flow angle (e.g., relative to the vertical direction V) directed away from the centerpoint C. Optionally, multiple vanes 136 may define flow angles directed away from the centerpoint C. In some such embodiments, separate vanes 136 define separate flow angles. For instance, the flow angles may generally and sequentially increase relative to the lateral direction L as the lateral distance between discrete vanes 136 increases relative to the centerpoint C. Thus, the flow angle defined by a first vane 136 proximal to the centerpoint C may be less than the flow angle defined by a second vane 136 distal from the centerpoint C (i.e., distal in comparison to the first vane 136). Advantageously, air from the air outlet grate 132 may be substantially dispersed within static chamber 120.

As shown, a vapor compression assembly 140 may be provided (e.g., to heat the air along airflow path 102 upstream from the air outlet grate 132). Optionally, airflow path 102 is defined as a closed loop. In turn, at least a portion of vapor compression assembly 140 may be disposed along the airflow path 102 (e.g., in fluid communication) between air inlet grate 130 and air outlet grate 132, as will be described in greater detail below.

Vapor compression assembly 140 includes a compressor 144 for selectively compressing the refrigerant, thus raising the temperature and pressure of the refrigerant. As is understood, as compressor 144 compresses refrigerant, refrigerant may generally be motivated along refrigerant flow path 142. Optionally, compressor 144 may for example be a variable speed compressor 144, such that the speed of the compressor 144 can be varied between zero (0) and one hundred (100) percent (e.g., as directed by a controller 138).

Vapor compression assembly 140 may further include a condenser 146, which may be disposed downstream from compressor 144 (e.g., in the direction of flow of the refrigerant within refrigerant flow path 142). Thus, condenser 146 may receive refrigerant from the compressor 144, and may condense the refrigerant by lowering the temperature of the refrigerant flowing therethrough due to, for example, heat exchange with ambient air.

Vapor compression assembly 140 further includes an evaporator 148 disposed downstream from the condenser 146. Additionally, an expansion device 150 (e.g., capillary tube, thermostatic expansion valve, electronic expansion valve, etc.) may be used to expand the refrigerant, thus further reduce the pressure of the refrigerant, leaving condenser 146 before being flowed to evaporator 148. Evaporator 148 generally is a heat exchanger that transfers heat from air passing over the evaporator 148 to refrigerant flowing through evaporator 148, thereby cooling the air and causing the refrigerant to vaporize. Outside of refrigerant flow path 142, evaporator 148 may condense water vapor within the air, thereby reducing the humidity or moisture content of air surrounding evaporator 148 or within an assembly casing 156.

From evaporator 148, refrigerant may flow back to and through compressor 144, which may be downstream from evaporator 148, thus completing refrigeration flow path.

As shown, evaporator 148 and condenser 146 may be mounted or disposed along the airflow path 102 between air inlet grate 130 and air outlet grate 132. In particular, evaporator 148 may be downstream from air inlet grate 130 and upstream from air outlet grate 132. Additionally or alternatively, condenser 146 may be downstream from air inlet grate 130 and upstream from air outlet grate 132. In some such embodiments, condenser 146 is mounted along airflow path 102 (e.g., in fluid communication) between evaporator 148 and air outlet grate 132. Thus, condenser 146 may be downstream from evaporator 148 while being upstream from air outlet grate 132 along the airflow path 102.

During operation of vapor compression assembly 140, air may flow through airflow path 102 from air inlet grate 130 and across evaporator 148. Due, for example, to the relatively cold temperature at evaporator 148, water vapor within airflow path 102 may condense and separate from the flowing air. After passing across evaporator 148, the dried air may pass across condenser 146, which may heat the dried air or otherwise raise the air temperature before such air is returned to static chamber 120 through air outlet grate 132.

A system blower or fan 134 may be used to force air through airflow path 102. In particular, system fan 134 may motivate air from air inlet grate 130 to air outlet grate 132, such as across evaporator 148 or condenser 146. As such, heated air is produced and directed to static chamber 120. Optionally, system fan 134 may be mounted along airflow path 102 (e.g., in fluid communication) between condenser 146 and air outlet grate 132. Thus, system fan 134 may be downstream from condenser 146 while being upstream from air outlet grate 132 along the airflow path 102. In certain embodiments, system fan 134 can be a variable speed fan. In other words, the speed of system fan 134 may be controlled or set anywhere between and including, for example, zero (0) and one hundred (100) percent. The speed of system fan 134 can be determined by, and communicated to, system fan 134 by controller 138. Separate from or in addition to system fan 134, a circulation fan 166 may be mounted within static chamber 120 to agitate or circulate air therein between air outlet grate 132 and air inlet grate 130 (e.g., as directed by controller 138).

Optionally, compressor 144 or expansion device 150 may be separated or isolated from airflow path 102 (e.g., within a machine compartment 152 defined by one or more compartment panels 154). During operation, air passing from air inlet grate 130 to air outlet grate 132 (e.g., across evaporator 148 or condenser 146) may thus be prevented from passing across compressor 144 or expansion device 150.

When assembled, vapor compression assembly 140 is attached (e.g., directly or indirectly) to cabinet 110. In some embodiments, vapor compression assembly 140 is mounted within a discrete assembly casing 156. For instance, assembly casing 156 may enclose at least a portion of vapor compression assembly 140. As shown, a casing sidewall 162 may extend about compressor 144, condenser 146, evaporator 148, and expansion device 150. Additionally or alternatively, system fan 134 may be enclosed within assembly casing 156. A casing base wall 158 and casing top wall 160 may be connected at opposite ends of casing sidewall 162. In optional embodiments, air inlet grate 130 or air outlet grate 132 may be mounted to casing top wall 160.

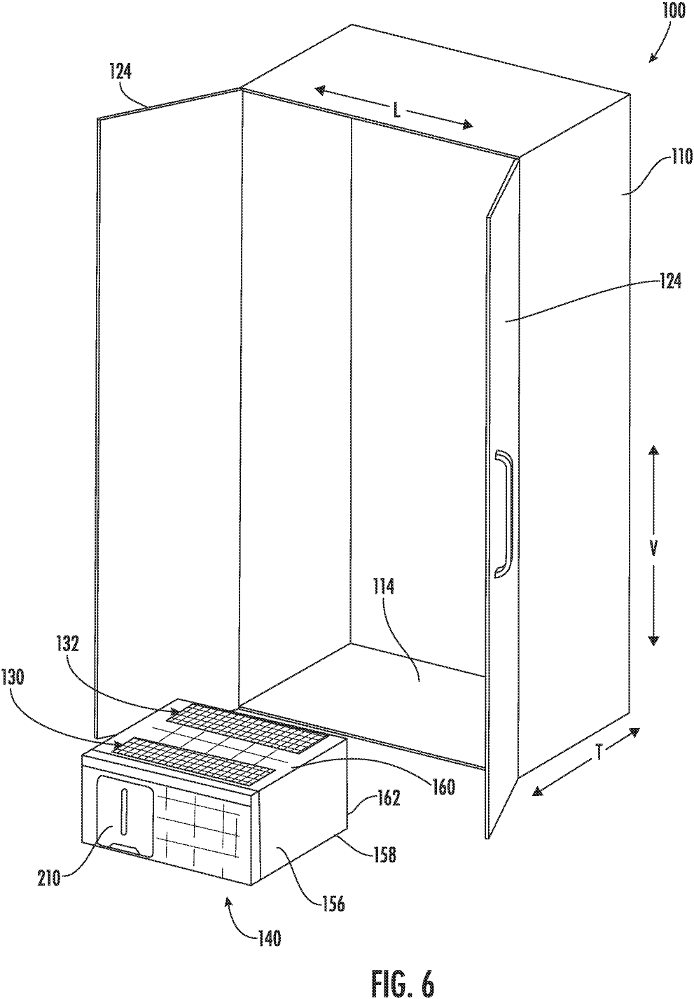

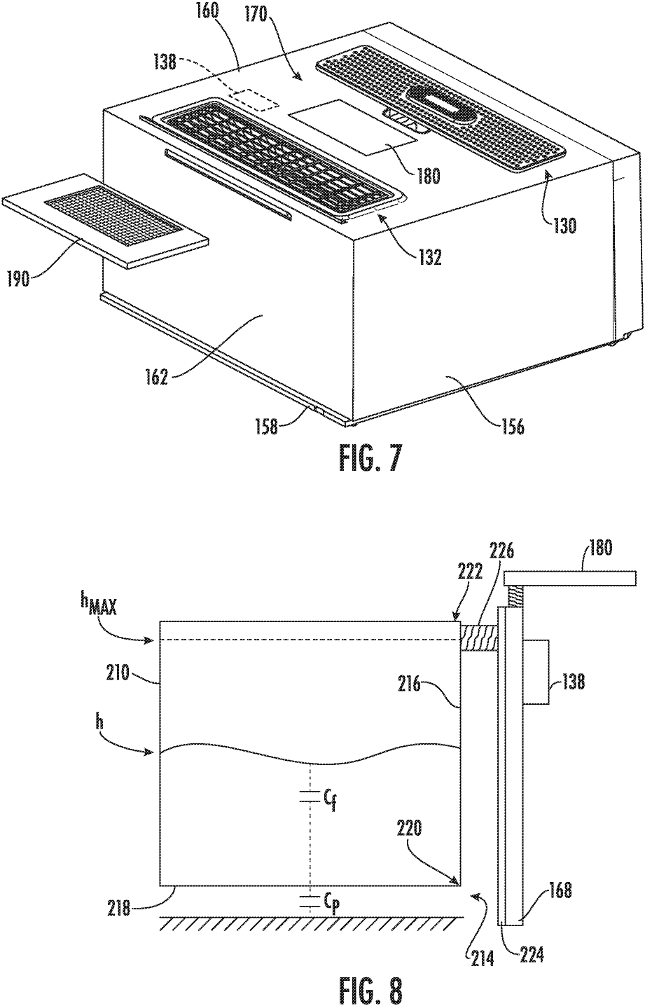

When assembled, vapor compression assembly 140 or assembly casing 156 may be positioned below (e.g., directly beneath) at least a portion of static chamber 120. For instance, assembly casing 156 may be mounted below cabinet 110 (e.g., to a common frame 164 with cabinet 110). In some such embodiments, lower internal wall 114 includes or defines at least a portion of casing top wall 160. In the illustrated embodiments of FIGS. 1 through 3, vapor compression assembly 140 and assembly casing 156 are mounted beneath the entirety of static chamber 120. By contrast, in the illustrated embodiments of FIG. 6, vapor compression assembly 140 and assembly casing 156 are selectively received within static chamber 120 (e.g., on top of lower internal wall 114).

Generally, vapor compression assembly 140 is in operative communication (e.g., wired or wireless communication) with a processing device or controller 138 having one or more circuit boards 168. For instance, controller 138 may be connected to one or more portions of vapor compression assembly 140 (e.g., compressor 144 or expansion device 150) via one or more wires, harness, connection busses, etc. to direct operation thereof. Additionally or alternatively, controller 138 may be connected to one or more further components of appliance 100 (e.g., system fan 134, circulation fan 166, etc.) to direct operation thereof. In certain embodiments, controller 138 is in communication with a user interface 170 having one or more selector inputs (e.g., knobs, buttons, touchscreen interfaces, etc.) or displays, (e.g., indicator lights or screens) attached to assembly casing 156 or cabinet 110. In optional embodiments, user interface 170 includes a capacitive touch panel 180 (e.g., attached to or on assembly casing 156). During use, certain signals generated in controller 138 may control or direction operation of vapor compression assembly 140 or other components in response to the user engagement at selector inputs. Other signals generated in controller 138 may be directed to the displays such that the displays present or projection information in response to such signals from controller 138.

As used herein, “processing device” or “controller” may refer to one or more microprocessors or semiconductor devices and is not restricted necessarily to a single element. The processing device can be programmed to operate dryer appliance 100. The processing device may include, or be associated with, one or more memory elements (e.g., non-transitive storage media) such as, for example, electrically erasable, programmable read only memory (EEPROM). The memory elements can store information accessible processing device, including instructions that can be executed by processing device. For example, the instructions can be software or any set of instructions that when executed by the processing device, cause the processing device to perform operations. In certain embodiments, the instructions include a software package configured to operate appliance 100.

In certain embodiments, appliance 100 further includes a temperature sensor 182 or a humidity sensor 184 (e.g., in operative communication with controller 138). A temperature sensor 182 or a humidity sensor 184 may, for example, be disposed on or within assembly casing 156 (e.g., spaced apart from static chamber 120) and may be configured to measure the temperature and relative humidity, respectively, of air flowing between air inlet grate 130 and air outlet grate 132. In some such embodiments, temperature sensor 182 or humidity sensor 184 is/are disposed upstream from evaporator 148 along the airflow path 102. In additional or alternative embodiments, temperature sensor 182 or humidity sensor 184 is/are mounted on or adjacent to evaporator 148. Moreover, is understood that sensors may be positioned at any other suitable location on or within assembly casing 156, and that additional sensors may be used throughout appliance 100 to measure temperature or humidity along the airflow path 102. Any suitable temperature sensor 182 and humidity sensor 184 may be used in accordance with the present disclosure.

In this regard, as discussed above, dryer appliance 100 may include controller 138, which is in communication with vapor compression assembly 140 and other components of dryer appliance 100. Controller 138 may additionally be in communication with temperature sensor 182 and humidity sensor 184. Thus, temperature sensor 182 and humidity sensor 184 may be used to control operation of the vapor compression assembly 140. For example, controller 138 may be configured to activate compressor 144 at full speed, activate it at partial speed, or deactivate it based on temperature or humidity signals received from sensors by controller 138.

In some embodiments, an ultraviolet (UV) purifier having a UV light source 188 is mounted within dryer appliance 100. In particular, UV light source 188 may be mounted adjacent to one or more portions of vapor compression assembly 140 (e.g., in communication with controller 138) to purify air flowing across vapor compression assembly 140. For instance, UV light source 188 may be mounted along airflow path 102 (e.g., in fluid communication) between air outlet grate 132 and evaporator 148. In other words, relative to the direction of air flowed across vapor compression assembly 140, UV light source 188 may be downstream from air outlet grate 132 and upstream from evaporator 148 (e.g., mounted within assembly casing 156). When assembled, UV light source 188 may be directed to a defined chamber or limited portion of the airflow path 102 to project UV-C light emissions to the air flowing therethrough, as would be understood. For instance, UV light source 188 may be directed downward at a position below air inlet grate 130. In turn, at least a portion of air passing across evaporator 148 (and thus later to static chamber 120) may be subjected to the UV-C light emissions projected from UV light source 188. Advantageously, odors or germs within dryer appliance 100 may be limited or otherwise prevented from accumulating within static chamber 120.

In optional embodiments, controller 138 is configured to initiate or direct activation of UV purifier 184 independently of vapor compression assembly 140. In particular, controller 138 may be configured to initiate one or more air treatment operations. As an example, controller 138 may be configured to initiate an air-purifying operation in which the UV purifier 184 is activated to emit UV-C light emissions while vapor compression assembly 140 is held in an inactive state. Optionally, system fan 134 may be activated to motivate air along the airflow path 102. As an additional or alternative example, controller 138 may be configured to initiate a refresh operation in which the UV purifier 184 and the vapor compression assembly 140 are both activated (e.g., simultaneously or in programmed succession). Thus, UV-C light emissions may be emitted from UV purifier 184 while air is heated or dried as is motivated across the vapor compression assembly 140 (e.g., by system fan 134). As a further additional or alternative example, controller 138 may be configured to initiate a dry-only operation in which the UV purifier 184 is held in an inactive state while vapor compression assembly 140 is activated.

In some embodiments, one or more porous air filters 190 having a filtration media (e.g., cellulose, fiberglass, carbon, etc.) are selectively received within appliance 100. For instance, a porous air filter 190 may be selectively mounted along airflow path 102 (e.g., in fluid communication) between air outlet grate 132 and air inlet grate 130. In the illustrated embodiments, air filter 190 is mounted between evaporator 148 or system fan 134 and air inlet grate 130 (e.g., within assembly casing 156). Thus, relative to the direction of air flowed across vapor compression assembly 140, air filter 190 may be downstream from evaporator 148 or assembly and upstream from air outlet grate 132. During use, air passing from evaporator 148 or system fan 134 and to static chamber 120, may be forced through air filter 190.

Along airflow path 102, liquid water or condensation may generally accumulate. For instance, the relatively cold operating temperatures of evaporator 148 may cause water vapor within the air to condensate on an outside surface of evaporator 148 (e.g., outside of refrigerant flow path 142). Over time, the liquid condensation may collect and fall from evaporator 148 (e.g., as motivated by air pressure or gravity). In some embodiments, a condensation path 192 is provided within appliance 100 to receive and guide the liquid condensation from the evaporator 148. Thus, the condensation path 192 may be disposed downstream from the evaporator 148 outside of the refrigerant flow path 142 (e.g., along at least a portion of the airflow path 102).

Condensation path 192 may extend between a first (e.g., upstream) end 194 and a second (e.g., downstream) end 196. In certain embodiments, the first end 194 is positioned below (e.g., directly beneath or offset by a guiding duct from) the evaporator 148 to collect liquid condensation falling from the evaporator 148. For instance, the first end 194 may be positioned within the airflow path 102. In additional or alternative embodiments, the second end 196 is positioned apart from the airflow path 102. Thus, the liquid condensation may be guided from the airflow path 102 at first end 194, through the condensation path 192, and outside of airflow path 102 to escape condensation path 192 at second end 196.

Generally, condensation path 192 may include any number of suitable pipes, ducts, funnels, or otherwise liquid-guiding structures between first end 194 and second end 196. In some embodiments, one or more condensation pumps 198 (e.g., rotary pumps, positive displacement pumps, etc.) may be disposed along the condensation path 192. Condensation pump 198 may be mounted, for instance, within assembly casing 156 or a sump defined within casing base wall 158. Additionally or alternatively, condensation pump 198 may be in communication with controller 138. During use, condensation pump 198 may be activated (e.g., as directed or at a pump speed selected by) controller 138 to motivate liquid condensation through condensation path 192 (e.g., from first end 194 to second end 196).

In exemplary embodiments, a water tank 210 is provided downstream from condensation path 192. Generally, water tank 210 has one or more tank walls (e.g., base tank wall or tank sidewall) that together define a tank volume that is at least partially enclosed while having at least one tank opening 212 to receive or collect at least a portion of the liquid condensation. When assembled, water tank 210 may be positioned at second end 196 in downstream fluid communication therewith. For instance, second end 196 may be disposed above water tank 210 (e.g., the defined tank volume). In additional or alternative embodiments, water tank 210 is slidably disposed on or received within assembly casing 156. Thus, water tank 210 may be selectively slid on or apart from assembly casing 156 (e.g., to empty the collected contents of the tank volume), as would be understood.

In optional embodiments, one or more volume sensors 214 are provided on or included with water tank 210 (e.g., in communication with controller 138). Generally, the volume sensor 214 may generate one or more signal correlated to or otherwise indicating a volume of liquid condensation or water within water tank 210 (e.g., in milliliters or as a relative percentage of tank volume). In the illustrated embodiments, water tank 210 includes at least one conductive plate (e.g., solid member formed from a conductive metal, such stainless steel, iron, aluminum, silver, copper, etc., including alloys thereof). As shown, a conductive sidewall plate 216 may extend along or as at least a portion of a corresponding sidewall of water tank 210. Specifically, conductive sidewall plate 216 extends along the vertical direction V from a bottom end 220 to a top end 222. Between bottom end 220 and top end 222, a tank capacitance circuit 224 may be connected to conductive sidewall plate 216. Specifically, capacitance circuit 224 may be electrically connected to conductive sidewall plate 216 via an intermediate conductor 226 (e.g., one or more wires, springs, conductive foam billets, etc.) such that capacitance circuit 224 is in electrical communication with conductive sidewall plate 216.

Generally, tank capacitance circuit 224 is mounted on or is provided in electrical communication with one or more circuit boards 168. Optionally, capacitance circuit 224 may be mounted on or included with a circuit board 168. In some such embodiments, the capacitance circuit 224 or circuit board 168 may be fixedly mounted within assembly casing 156. Thus, water tank 210 may selectively move into and out of electrical communication with capacitance circuit 224 or circuit board 168. Notably, intermediate conductor 226 may prevent the water tank 210 from removed (e.g., from assembly casing 156) or facilitate the contact of the intermediate conductor 226 with the conductive sidewall plate 216 of the water tank 210 when the water tank 210 is returned (e.g., to assembly casing 156).

Additionally or alternatively, tank capacitance circuit 224 may share a common circuit board 168 (e.g., as or as part of controller 138) with a capacitive touch panel 180. Thus, the common circuit board 168 may be in electrical communication with both capacitive touch panel 180 and tank capacitance circuit 224. In some such embodiments, capacitive touch panel 180 is mounted adjacent to (e.g., above water tank 210, such as on the casing top wall 160 of assembly casing 156) and notably minimizes number of necessary parts or total volume for assembly casing 156 or appliance 100, generally.

In exemplary embodiments, volume sensor 214 functions by detecting the parasitic capacitance between the bottom of the water tank 210 and the ground and the electrostatic capacitance between the water or liquid condensation and the bottom of the water tank 210. In some such embodiments, a conductive pole plate 218 is disposed at a bottom of the water tank 210, and a conductive sidewall plate 216 is disposed on an inner wall side of the water tank 210. In some embodiments, conductive pole plate 218 is fixed relative to conductive sidewall plate 216. Additionally or alternatively, conductive pole plate 218 or conductive sidewall plate 216 may be integrally formed on the water tank 210, that is, the body of the water tank 210 serves as a conductor, and the bottom of the water tank 210 serves as a capacitor. Thus, during use, conductive sidewall plate 216 may electrically communicate (i.e., be in electrical communication) with the conductive pole plate 218 and the intermediate conductor 226, and the side faces are in contact with water.

When assembled, the conductive pole plate 218 is insulated from the ground. In certain embodiments, bottom end 220 of the conductive sidewall plate 216 is connected to the conductive pole plate 218. Optionally, top end 222 may be connected the intermediate conductor 226 (e.g., at the top of the water tank 210), which is connected to the capacitance circuit 224 or circuit board 168. In some embodiments, a capacitor is formed between the conductive pole plate 218 and the ground, and the capacitance value between the conductive pole plate 218 and the ground is taken as a lead to facilitate measurement, and the capacitance value between the conductive pole plate 218 and the ground is detected by charged and discharge time (e.g., by controller 138), and then calculate the water volume or level.

In optional embodiments, controller 138 is configured to initiate or direct a volume-measurement operation comprising one or more of the below steps one through four.

Step one may include detecting capacitance of the water tank 210 in an empty state. For instance, in order to avoid the change of the height of the different devices and the influence of the change of the ambient temperature and humidity around the water tank 210, the capacitance value of the empty water tank 210 may be detected before each water level (e.g., at an empty state in which the tank volume of water tank 210 is free of any liquid water). The conductive pole plate 218 may completely discharged, and then charged. Subsequently, the capacitance Cₚ on the intermediate conductor 226 may be detected. After the detection is completed, the capacitor Cₚ is completely discharged again. At this time, the capacitance Cₚ is a parasitic capacitance existing between the conductive pole plate 218 and other conductors before the ground.

Step two may include detecting the capacitance of the water tank 210 with water, after water in the water tank 210, charging the conductive pole plate 218 to detect the capacitance Cₜ of the intermediate conductor 226. At this time, the capacitance Cₜ includes the capacitance Cₚ, and the water and the conductive pole plate 218 a capacitor C_{f}, wherein the capacitor Cₚ is connected in parallel with the capacitor C_{f}, that is, Cₜ= (Cₚ+C_{f});

Step three may include measuring the current water level height h. For instance, the difference between Cₜ and Cₚ may be determined to then calculate the current water level height h. In some such embodiments, the water level is measured according to the following formula:

$${C}_{p} = \frac{ε 1 S}{d} , {C}_{f} = \frac{ε 2 S}{h} ,$$

which is

$${C}_{t} = {C}_{p} + {C}_{f} = \frac{ε 1 S}{d} + \frac{ε 2 S}{\text{h}}$$

Thus derived

$$h = \frac{ε 2 S}{{C}_{t} - {C}_{p}}$$

Wherein, ε1 is the dielectric constant of the air between the conductive pole plate 218 and the ground, ε2 indicating the dielectric constant of water or liquid condensation, S is the surface area of the conductive pole plate 218 to the ground, and d is the distance from the conductive pole plate 218 to the ground.

Step four may include determining a maximum volume has been reached. For instance, a preset height hₘₐₓ of a maximum volume may be recorded in the controller 138. The measured height h may then be compared with the preset height hₘₐₓ. Optionally, the value of hₘₐₓ is lower than the actual height of the water tank 210. When h is equal to or greater than hₘₐₓ, a maximum volume state may be determined. In some such embodiments, controller 138 issues a warning to user (e.g., as an audio or visual alert). As an example, when the water level reaches a maximum volume, the controller 138 can control a buzzer or display to issue a warning to manually pour water from water tank 210 (e.g., after removing water tank 210 from casing 156).

Advantageously, the present water tank 210 and volume sensor 214 may provide a relatively simple and accurate assembly for detecting accumulated liquid condensation (e.g., within water tank 210).

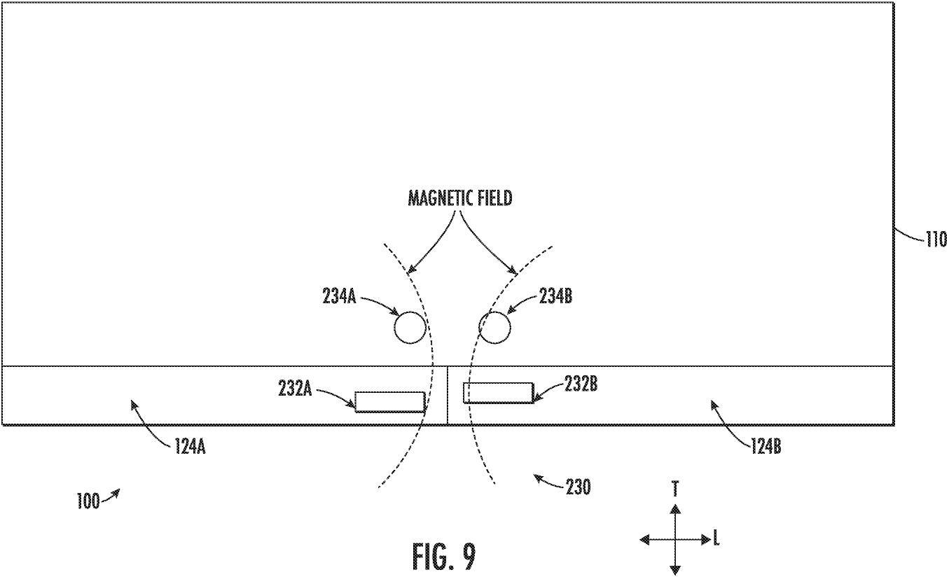

Referring generally to FIGS. 9 through 11, in optional embodiments, a protective cutoff assembly 230 is provided (e.g., in operative communication with controller 138) to halt operation of one or more components of appliance 100 (e.g., vapor compression assembly 140, UV purifier 184, system fan 134, etc.) in response to opening one or more doors to static chamber 120.

In some embodiments, the protective cutoff assembly 230 may generally detect an open or closed state of a door and outputting a corresponding position signal (e.g., to controller 138 which may be configured to receive the position signal). In response to receiving the position signal, controller 138 may direct one or more components to halt or otherwise prevent operation.

In certain embodiments, the protective cutoff assembly 230 includes a Hall sensor disposed in the cabinet 110 (e.g., above or below static chamber 120) and a magnet (e.g., a magnetic strip or a magnetic coating) disposed on at least one chamber door, the Hall sensor is disposed facing the magnet, and the Hall sensor determines the cabinet 110 by detecting a change in the strength of the surrounding magnetic field (e.g., when the door is opened or closed). The Hall sensor is disposed in the cabinet 110 and fixed in position, and the magnet is away from the cabinet 110 as the chamber door is opened. Thus, the magnetic field of the magnet is away from the Hall sensor during the opening process, and the Hall sensor senses the surrounding magnetic field. It is weakened during the opening of the chamber door to judge that the chamber door is open.

Referring especially to FIG. 9, which is a first embodiment of the protective cutoff assembly 230, the protective cutoff assembly 230 includes two discrete magnets 232A, 232B and two discrete corresponding Hall sensors 234A, 234B. The first magnet 232A is mounted in a first chamber door 124A and the second magnet 232B is mounted in a second chamber door 124B. A first Hall sensor 234A corresponds to the first magnet 232A, and a second Hall sensor 234B corresponds to the second magnet 232B. In the present embodiment, the first magnet 232A is mounted at a position where the first chamber door 124A is adjacent to the second chamber door 124B, and the second magnet 232B is mounted at a position where the second chamber door 124B is adjacent to the first chamber door 124A.

When the first chamber door 124A is opened, the movement of the first magnet 232A causes the first Hall sensor 234A to detect a change in the magnetic field, thereby issuing a position signal for opening the first chamber door 124A to the controller 138. In response to controller 138 receiving the position signal, operation of UV purifier 184, vapor compression assembly 140, or system fan 134 may be halted. Similarly, when the second chamber door 124B is opened, the movement of the second magnet 232B causes the second Hall sensor 234B to detect a change in the magnetic field, thereby issuing a position signal for opening the second chamber door 124B to the controller 138. In response to controller 138 receiving the position signal, operation of UV purifier 184, vapor compression assembly 140, or system fan 134 may be halted.

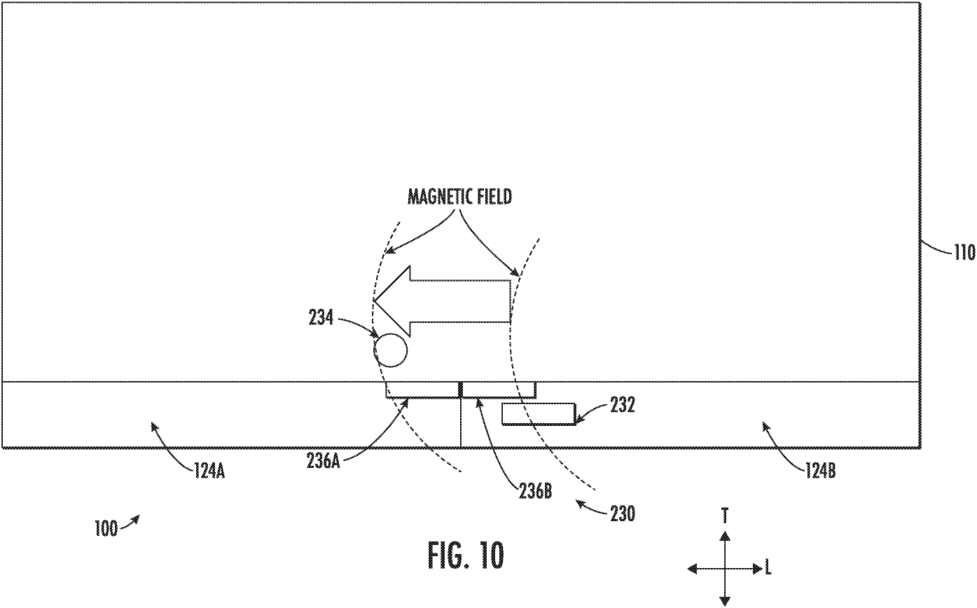

Referring to FIG. 10, there is shown a third embodiment of the protective cutoff assembly 230 is shown. The protective cutoff assembly 230 includes a magnet 232 and a single Hall sensor 234. The protective cutoff assembly 230 further includes two discrete conductive billets (e.g., a first conductive billet 236A and a second conductive billet 236B). In some such embodiments, the first conductive billet 236A and the second conductive billet 236B are mounted in the first chamber door 124A and the second chamber door 124B, respectively.

The first conductive billet 236A and the second conductive billet 236B are respectively mounted at ends of the first chamber door 124A and the second chamber door 124B, thereby forming a magnetic field having a stable strength between the first conductive billet 236A, the second conductive billet 236B, and the magnet 232. Thus, when the first chamber door 124A or the second chamber door 124B is opened, the corresponding magnetic field strength changes and is sensed by the single Hall sensor 234, and the single Hall sensor 234 feeds back information to the controller 138.

In exemplary embodiments, the single Hall sensor 234 is mounted on a side corresponding to the first chamber door 124A or the second chamber door 124B, and the magnet 232 is mounted on the other side corresponding to the first chamber door 124A or the second chamber door 124B. In some embodiments, the first conductive billet 236A or the second conductive billet 236B is a metal iron piece. In other embodiments, the first conductive billet 236A or the second conductive billet 236B may be provided as a workpiece of cobalt or nickel.

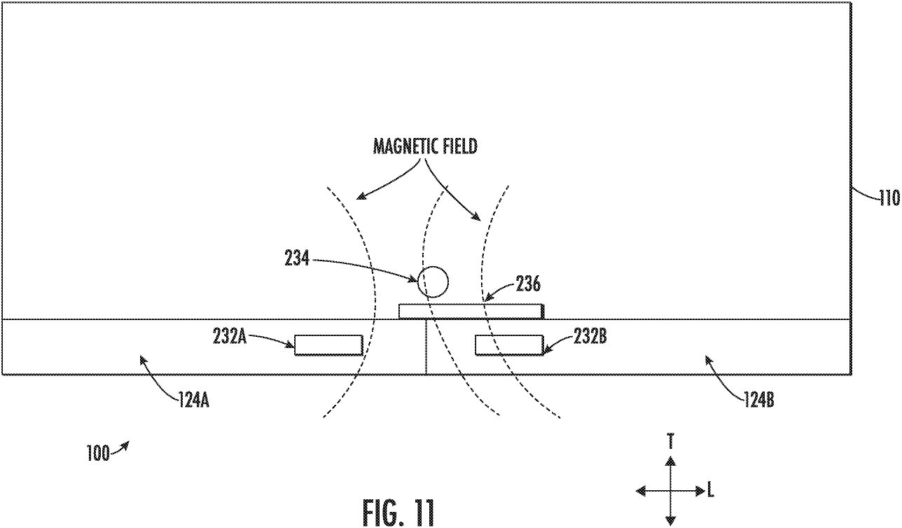

Referring especially to FIG. 11, a second embodiment of the protective cutoff assembly 230 is shown. The protective cutoff assembly 230 includes two discrete magnets 232A, 232B and a single Hall sensor 234. As shown, a first magnet 232A is mounted in the first chamber door 124A and a second magnet 232B is mounted in the second chamber door 124B. In some such embodiments, the single Hall sensor 234 is mounted on one side of the first chamber door 124A and the second chamber door 124B, and the protective cutoff assembly 230 further includes a conductive billet 236. The conductive billet 236 is located between the single Hall sensor 234 and the magnets 232A, 232B.

In some embodiments, the conductive billet 236 is a metal iron piece. In other embodiments, the conductive billet 236 may be provided as a workpiece of cobalt or nickel. As shown, the single Hall sensor 234 may be mounted adjacent to the closed door slit of the first chamber door 124A and the second chamber door 124B. Additionally or alternatively, the conductive billet 236 is mounted in the cabinet 110 and located between the single Hall sensor 234, the first magnet 232A and the second magnet 232B.

Generally, the conductive billet 236 is located within the magnetic field of the first magnet 232A and the second magnet 232B and is magnetized. When the first chamber door 124A or the second chamber door 124B is opened, the corresponding magnetic field strength changes and is sensed by the single Hall sensor 234, and the single Hall sensor 234 feeds information back to the controller 138.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A drying system defining an airflow path, the drying system comprising:
a cabinet defining a static chamber along the airflow path for the receipt of articles therein,
an air outlet grate upstream from the static chamber along the airflow path to direct air thereto;
an air inlet grate downstream from the static chamber along the airflow path to direct air therefrom;
a door attached to the cabinet to selectively restrict access to the static chamber;
a vapor compression assembly attached to the cabinet and defining a refrigerant flow path spaced apart from the static chamber, the vapor compression assembly comprising
a compressor to selectively compress refrigerant within the refrigerant flow path,
an evaporator in fluid communication with the refrigerant flow path, the evaporator being mounted along the airflow path between the air inlet grate and the air outlet grate, and
a condenser in fluid communication with the refrigerant flow path, the condenser being mounted along the airflow path between the evaporator and the air outlet grate; and
an ultraviolet light source mounted along the airflow path between the air outlet grate and the evaporator.

2. The drying system of claim 1, further comprising:
a condensation path disposed downstream from the evaporator outside of the refrigerant flow path to receive a liquid condensation therefrom, the condensation path extending from a first end positioned below the evaporator to a second end positioned apart from the airflow path; and
a water tank positioned at the second end of the condensation path to receive the liquid condensation.

3. The drying system of claim 2, wherein the water tank comprises a conductive sidewall plate extending from a bottom end to a top end, and wherein the drying system further comprises a tank capacitance circuit in electrical communication with the conductive sidewall plate.

4. The drying system of claim 3, further comprising:
a user interface comprising a capacitive touch panel adjacent to the water tank to receive a user input; and
a common circuit board in electrical communication with the capacitive touch panel and the tank capacitance circuit.

5. The drying system of claim 2, further comprising a condensation pump disposed along the condensation path to motivate the liquid condensation from the first end to the water tank.

6. The drying system of claim 1, wherein the air inlet grate is positioned rearward from the air outlet grate along a transverse direction and defines a laterally-expansive airflow passage therethrough along a vertical direction.

7. The drying system of claim 1, further comprising a temperature sensor mounted to the vapor compression assembly apart from the static chamber.

8. The drying system of claim 1, further comprising a humidity sensor mounted to the vapor compression assembly apart from the static chamber.

9. The drying system of claim 1, further comprising an assembly casing slidably positioned on the cabinet at a lower end of the static chamber, wherein the vapor compression assembly is enclosed within the assembly casing.

10. The drying system of claim 9, wherein the assembly casing comprises a casing top wall positioned above the vapor compression assembly, and wherein the air outlet grate and the air inlet grate are mounted to the casing top wall.

11. A drying system defining an airflow path, the drying system comprising:
a cabinet defining a static chamber along the airflow path for the receipt of articles therein,
an air outlet grate upstream from the static chamber along the airflow path to direct air thereto;
an air inlet grate downstream from the static chamber along the airflow path to direct air therefrom;
a door attached to the cabinet to selectively restrict access to the static chamber;
a vapor compression assembly attached to the cabinet and defining a refrigerant flow path, the vapor compression assembly comprising
a compressor to selectively compress refrigerant within the refrigerant flow path,
an evaporator in fluid communication with the refrigerant flow path, the evaporator being mounted along the airflow path between the air inlet grate and the air outlet grate, and
a condenser in fluid communication with the refrigerant flow path, the condenser being mounted along the airflow path between the evaporator and the air outlet grate;
a condensation path disposed downstream from the evaporator outside of the refrigerant flow path to receive a liquid condensation therefrom, the condensation path extending from a first end positioned below the evaporator to a second end positioned apart from the airflow path;
a water tank positioned at the second end of the condensation path to receive the liquid condensation, the water tank comprising a conductive sidewall plate extending from a bottom end to a top end; and
a tank capacitance circuit in electrical communication with the conductive sidewall plate.

12. The drying system of claim 11, further comprising
a user interface comprising a capacitive touch panel adjacent to the water tank to receive a user input; and
a common circuit board in electrical communication with the capacitive touch panel and the tank capacitance circuit.

13. The drying system of claim 11, further comprising a condensation pump disposed along the condensation path to motivate the liquid condensation from the first end to the water tank.

14. The drying system of claim 11, wherein the air inlet grate is positioned rearward from the air outlet grate along a transverse direction and defines a laterally-expansive airflow passage therethrough along a vertical direction.

15. The drying system of claim 11, further comprising a temperature sensor mounted to the vapor compression assembly apart from the static chamber.

16. The drying system of claim 11, further comprising a humidity sensor mounted to the vapor compression assembly apart from the static chamber.

17. The drying system of claim 11, further comprising an assembly casing slidably positioned on the cabinet at a lower end of the static chamber, wherein the vapor compression assembly is enclosed within the assembly casing.

18. The drying system of claim 17, wherein the assembly casing comprises a casing top wall positioned above the vapor compression assembly, and wherein the air outlet grate and the air inlet grate are mounted to the casing top wall.
